(19) United States
(12) Patent Application Publication
(10) Pub. No.: US 2003/0166609 A1
(43) Pub. Date: Sep. 4, 2003

(54) Medicine containing polysacccharide substances for activating apoptosis

(76) Inventors: Jean-Claude Yvin, Saint Malo (FR); Andre Patrick Arrigo, Pers Jussy (FR)

Correspondence Address: John S Pratt, Kilpatrick Stockton, 1100 Peachtree Street, Suite 2800, Atlanta, GA, 30309-4530, US

(21) Appl. No.: 10/258,840

(22) PCT Filed: Apr. 27, 2001
(86) PCT No.: PCT/FR01/01323

(30) Foreign Application Priority Data
Apr. 27, 2000 (FR) 00/05375

(51) Int. Cl. A61K 31/737; A61K 31/715

(52) U.S. Cl. 514/54; 514/61

(57) ABSTRACT

The invention concerns the use of at least a polysaccharide substance having at least five saccharide units and comprising at least some single-unit motifs thereof, at least a substituent bearing at least a negative charge selected among the group comprising in particular the sulphate, acetate, phosphate, phosphonate groups, for preparing a medicine for activating apoptosis by increasing the number of apoptosis receptors at the cell surface, said receptors being those of the group comprising Fas, TNF, TRAIL, CD40, preferably Fas and TNF, receptors.

The invention relates to a medicine based on certain polysaccharide substances for treating apoptosis dysfunction, and more particularly for activating apoptosis.

It consists in using these polysaccharide substances to prepare a medicine for activating apoptosis by increasing the number of apoptosis receptors at the surface of the cells in which it is desired to induce apoptosis.

It is recalled that the term “apoptosis” denotes programmed cell death or cell suicide.

This death corresponds to self-elimination of cells according to a defined program.

It reveals itself, initially, through bulges in the plasma membrane, these bulges being accompanied by a structural change in the membrane, and then through a loss of volume of the cell, which appears to contract and to collapse in on itself.

The nucleus condenses and the DNA is cleaved into small fragments (Raff, Nature, 356, 397, 1992; Bortner et al., “Trends in Cell. Biol.” 5, 21, 1995).

In vivo, the cell undergoing apoptosis is recognized by macrophages which will phagocytose it and eliminate it without any inflammatory process.

Still in vivo, apoptosis is widely used by living organisms to control cell populations, in particular lymphocytes subsequent to their activation.

Moreover, during the development of organisms, apoptosis plays a fundamental role in the elimination of unnecessary embryonic tissues (lizard tail, rudiment of the genital organs of one sex or the other) and in the pattern of the organism (elimination of interdigital webs between the future fingers, etc.).

Some compounds which are present in living organisms specifically induce an apoptotic phenomenon. Thus, for example in mammals, the binding of the Fas ligand to the Fas membrane-bound receptor, which is also referred to as APO-1 or CD95, specifically induces an apoptosis; this apoptosis is used by the living organism to control lymphocyte populations, in particular T lymphocyte populations.

The abovementioned receptor and ligand represent an extremely advantageous physiological system which is involved in the specific elimination of cells which are no longer desired in the organism.

Mention may be made in particular of cell elimination during the maturation and activation of T lymphocytes. In fact, the Fas system, i.e. Fas ligand/Fas receptor, plays a fundamental role in immune system homeostasis.

The Fas receptor is a member of a family of proteins which act as cell surface receptors and which also comprise the TNF (tumoral necrosis factor), TRAIL (Tumor Related Apoptosis Induced Ligand), CD40 and NGF (nerve growth factor) receptors.

The Fas receptor is expressed in many cells; it is thought to accumulate in the Golgi apparatus.

The mechanism by which the Fas system induces cell death is unknown, but involves the activation of proteases which are known under the name ICE-like (interleukin-1 beta-converting enzyme-like) or caspases.

It may be noted that the Fas ligand can be secreted by cells in order to induce their own suicide; however, given that this ligand is also found at the surface of activating cells, these cells will, as a result, induce the suicide of target cells by simple contact.

Once activated, the Fas receptor interacts with many intracellular proteins so as to transmit the apoptosis-triggering signal.

In vitro, other means exist for inducing apoptosis, for example by inhibiting the activity of certain kinases, and in particular kinase C; in this case, use may be made of staurosporine.

This product is very effective for inducing cell death by apoptosis.

It should, however, be noted that the transduction of the signals induced by staurosporine is different from that involving the Fas receptor.

However, while the means for activating apoptosis are different, the execution of the death program induced by these two methods of activation is equivalent and characterized by activation of the caspase cascade and dysfunction of mitochondria, which release compounds (for example cytochrome C) which will promote the programmed destruction of the cell. This phenomenon is energy dependent, but does not require the synthesis of new proteins. In fact, in a cell, everything is ready for it to carry out its own destruction.

In vivo, regulation of the apoptotic phenomenon is of great importance.

In fact, many pathologies are associated with a dysfunction thereof, in particular with a deficiency therein.

Mention may be made, for example, of the case of auto-immune diseases in which apoptosis is deficient and that of the accumulation of cancer cells in which apoptosis would appear to depend on the Fas system (“Green”, Science, vol. 278, 1246, 1997).

It is known that one portion of apoptosis receptors is present at the cell surface and that another portion is located inside the cells; it is also known that only the apoptosis receptors present at the cell surface enable apoptosis to be triggered, the receptors located inside the cells having no apoptotic activity.

In addition, the applicant company, to its credit, has found that polysaccharide substances having at least five saccharide units and comprising, on at least some of their individual units, at least one substituent carrying at least one negative charge, chosen from the group comprising in particular sulfate, acetate, phosphate and phosphonate groups, are capable of inducing an increase in the number of apoptosis receptors at the surface of cells in which apoptosis must be activated.

A subject of the invention is therefore the use of a polysaccharide substance having at least five saccharide units and comprising, on at least some of its individual units, at least one substituent carrying at least one negative charge, chosen from the group comprising in particular sulfate, acetate, phosphate and phosphonate groups, for preparing a medicine for activating apoptosis by increasing the number of apoptosis receptors at the cell surface, said receptors being those of the group comprising Fas, TNF, TRAIL and CD40 receptors, preferably Fas receptors.

According to an advantageous embodiment, the polysaccharide substance is chosen from the group comprising:

the oligosaccharides derived, by enzymatic or chemical process, from the polymers of the group comprising β-1,3 glucans optionally comprising β-1,6 branchings, and comprising, on at least some of their individual units, at least one substituent carrying at least one negative charge, chosen from the group comprising in particular sulfate, acetate, phosphate and phosphonate groups,

the oligosaccharides derived, by enzymatic or chemical process, from sulfated galactans, in particular carrageenans, agars and porphyrans.

According to another advantageous embodiment, the polysaccharide substance corresponds to the formula:

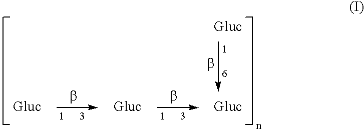

in which Gluc represents glucose, n represents an integer from 2 to 50, preferably from 5 to 10, and in which the number of branches varies from 0 to 3 per repeat unit, and comprises, on at least some of its individual units, at least one substituent carrying at least one negative charge, chosen from the group comprising in particular sulfate, acetate, phosphate and phosphonate groups.

According to another advantageous embodiment, the polysaccharide substance is a repeat disaccharide corresponding to the formula:

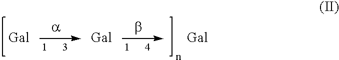

in which Gal represents galactose and n represents an integer from 2 to 50, preferably from 2 to 20, at least some of the repeat disaccharides of formula (II) possibly comprising one or more sulfate groups.

According to another particularly advantageous embodiment, the polysaccharide substance is a dextran comprising, on at least some of its individual units, at least one substituent carrying at least one negative charge, chosen from the group comprising in particular sulfate, acetate, phosphate and phosphonate groups, preferably a sulfated dextran, and advantageously a sulfated dextran marketed by the company SIGMA.

According to another advantageous embodiment, the polysaccharide substance is a natural polysaccharide of the iota-carrageenan type extracted from red algae and marketed by the company HERCULES, which has the formula

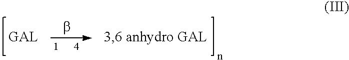

in which:

GAL represents galactose,

3,6 anhydro GAL represents 3,6 anhydrogalactose linked via a β1-4 linkage, and

n represents an integer from 2 to 300, preferably from 2 to 200,

the repeat disaccharides possibly carrying 2 sulfate esters.

More particularly, the invention is directed toward the use of one of the abovementioned polysaccharide substances, for producing a medicine for activating apoptosis by increasing the number of apoptosis receptors at the cell surface in the treatment of diseases belonging to the group of cancers, of auto-immune diseases and asthma.

According to a particularly advantageous embodiment, the invention relates to the use of the abovementioned polysaccharide substances, for preparing a medicine provided in the form of an aerosol, in particular for the treatment of asthma.

According to another particularly advantageous embodiment, the invention relates to the use of one of the abovementioned polysaccharide substances, in combination with an anti-apoptosis receptor antibody, for preparing a medicine for activating apoptosis by increasing the number of apoptosis receptors at the cell surface.

According to another advantageous embodiment, a subject of the invention is the use of the abovementioned polysaccharides, for preparing a medicine which is made to comprise adjuvants chosen depending on the method of administration and of the dose selected.

In particular, especially in the case of the treatment of asthma, the adjuvants are chosen from those which make it possible to obtain a medicine which can be administered by inhalation.

In the case of apoptosis which is induced by ultra-violet radiation (UV A and B) , which is Fas receptor-dependent, as described in Aragane et al. in J. Cell Biol. 140, 171-182 (1998), Kulms et al., Proc. Natl. Acad. Sci. USA 96, 7974-7979 (1999), Schwarz et al., J. Immunol. 160, 4262-4270 (1998), the adjuvants are chosen from those which make it possible to obtain a medicine for topical application which can, for example, be incorporated into an ointment.

According to another embodiment of the invention, the polysaccharide substance is used in the form of a physiological solution with a concentration of 0.005 mg/ml to 0.5 mg/ml, preferably of 0.01 to 0.2 mg/ml and even more preferentially of 0.02 mg/ml.

It ensues that the medicine obtained through the use in accordance with the invention of the abovementioned polysaccharide substances is generally provided in the form of a solution intended to be administered in external form for topical application (ointment), in the form of an aerosol or in injectable form.

The invention will be more clearly understood from the further description which follows and the examples which are in no way limiting but correspond to advantageous embodiments.

In the experiments which will be described below, work was carried out on cell cultures in which an apoptotic process was triggered using the Fas system; in the context of these experiments, the increase in the number of Fas or TNF receptors at the cell surface, obtained with the polysaccharide substances defined above, was determined qualitatively.

Still within the context of the experiments, firstly, the doses of polysaccharide substances having to be employed in the treatment of diseases using the medicines obtained using the abovementioned polysaccharide substances and, secondly, the duration of activity of these polysaccharide substances, in other words the duration of the effect sought, namely an increase in the number of apoptosis receptors at the surface of the cells in which it is desired to activate apoptosis via the endogenous (the host's) Fas ligand, were determined.

### EXAMPLE 1

The substance studied is dextran sulfate.

Work was carried out on a culture of immortalized human cells consisting of T lymphocytes (of the Jurkat type).

The medium used for culturing the Jurkat-type cells is that marketed by the company Life Technologies under the name “RPMI 1640 Medium”; this medium is described by Moore et al. in the publication “A.M.A.” 199, 519 (1967).

10⁶ T lymphocytes of the Jurkat type were suspended in 5 ml of this medium. The culturing was carried out in an incubator at a temperature of 37° C. and in an atmosphere containing 5% CO₂.

After incubation for 24 hours, the cells multiplied to reach 2×10⁶ cells.

These cells are recovered by centrifugation, and are washed in an isotonic PBS buffer. A further incubation of these 2×10⁶ cells is then carried out in 1 ml of the abovementioned medium at 4° C. and in an atmosphere containing 5% CO₂, for 45 minutes in the presence of an anti-Fas receptor, sold under the catalog number 05-201 by Euromedex, diluted to 1/50 in PBS containing 1% of bovine serum albumin (BSA).

At the end of this incubation, the cells are collected and are washed with the isotonic PBS buffer. After washing, a further incubation of the cells is carried out for 45 minutes at 4° C. in the presence of an antibody against hamster immunoglobulins (IgGs) (produced in the goat) which is coupled to fluorescein isothiocyanate, diluted to 1/50 in PBS containing 1% of BSA (marketed by Euromedex, No. AP128F).

Controls were prepared by incubating the T lymphocytes of the Jurkat type only with the second antibody (goat anti-hamster IgG coupled to fluorescein isothio-cyanate).

The increase in the number of Fas receptors at the surface of the cells, under the action of dextran sulfate, is then determined.

Consequently, the fluorescence of the cells (which is proportional to the number of receptors present at the surface) is determined in the absence of dextran sulfate and in the presence of dextran sulfate for a number of solutions at various concentrations. The ratio of the fluorescence in the absence of dextran sulfate to the fluorescence with dextran sulfate is then calculated. In the following text, the result of this calculation is called “ratio”.

Various assays are carried out by introducing into the culture medium, at time t=0 of the experiment, dextran sulfate at the following doses: 0.2 mg/ml, 0.1 mg/ml, 0.02 mg/ml and 0.002 mg/ml; the dextran sulfate is that marketed by the company Sigma.

After various times of incubation with the dextran sulfate, the presence of Fas receptors at the surface of the cells is detected by fluorescence. This fluorescence is induced by the fluorescein isothio-cyanate coupled to the anti-Fas receptor antibodies which are attached to the Fas receptors, and was measured by passing a cell sample containing 10⁴ cells through a device such as those which function by flow cytometry, in this case that marketed by the company Beckton Dickinson under the name “FACS Scan Flow Cytometer”. The intensity of the fluorescence, which is expressed in arbitrary units, therefore depends on the number of Fas receptors present at the surface of the cells making up the sample.

For each sample, the curve representing the variation in fluorescence as a function of said number of cells having receptors at their surface is constructed on a graph on which the fluorescence (arbitrary units) is recorded on the X axis and the number of cells (to within one multiplication factor) having receptors at their surface is recorded on the Y axis; this curve reflects a Gaussian-type distribution of the fluorescence.

In fact, in the population of cells present in a given sample, not all the cells have the same fluorescence, which comes from the fact that they do not all have the same number of receptors at their surface.

The various assays carried out make it possible to classify the cell populations as a function of the fluorescence, i.e. as a function of the number of receptors present at the surface of the cells.

The different maximum fluorescence values for each assay, which reflect the different numbers of receptors present at the surface, depend on the active products used in the various assays. The more effective the active principle used, the higher the number of surface receptors, and the higher the maximum fluorescence value.

In FIGS. 1 to 4, curve C₁ represents the Gaussian distribution of the fluorescence of the sample of control cells (without dextran sulfate) (sample 1).

In FIG. 1, curve C₂ represents the Gaussian distribution of the fluorescence of the cell sample for which the incubation was carried out in the presence of 0.2 mg/ml of dextran sulfate (sample 2).

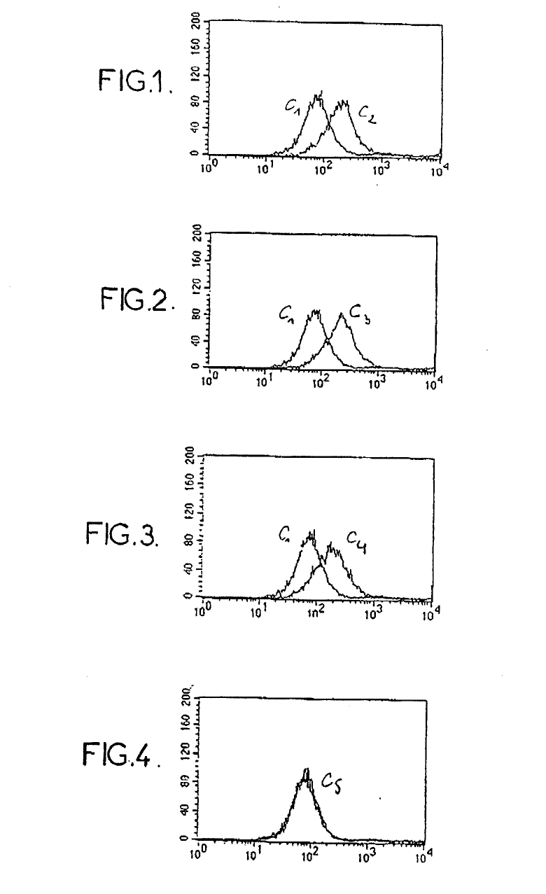

In FIG. 2, curve C₃ represents the Gaussian distribution of the fluorescence of the cell sample for which the incubation was carried out in the presence of 0.1 mg/ml of dextran sulfate (sample 3).

In FIG. 3, curve C₄ represents the Gaussian distribution of the fluorescence of the cell sample for which the incubation was carried out in the presence of 0.02 mg/ml of dextran sulfate (sample 4).

In FIG. 4, curve C₅ represents the Gaussian distribution of the fluorescence of the cell sample for which the incubation was carried out in the presence of 0.002 mg/ml of dextran sulfate (sample 5).

The maximum values (value of the peak of each of curves C₁ to C₅) and also the corresponding ratio are given in table I below:

TABLE I

| | |
|---|---|
| Concentration of | Fluorescence |

| dextran sulfate | Value measured | Ratio |
|---|---|---|
| 0 (curve C₁) | 80 | 1 |
| 0.2 mg/ml (curve C₂) | 250 | 3.13 |
| 0.1 mg/ml (curve C₃) | 250 | 3.13 |
| 0.02 mg/ml (curve C₄) | 200 | 2.50 |
| 0.002 mg/ml (curve C₅) | 90 | 1.13 |

On examining the results given in table I, it appears that the minimum concentration of dextran sulfate is 0.02 mg/ml, that at a greater dilution the effect is too diminished, and that a higher concentration does not enhance the effect.

Another series of assays was then carried out in order to measure the duration of the effect consisting of an increase in the number of Fas receptors at the cell surface caused by 0.02 mg/ml of dextran sulfate.

To do this, the dose of dextran sulfate was introduced into the culture medium and the number of receptors at the surface was measured using the abovementioned FACS Scan Flow Cytometer device, immediately after introduction, and then 6 h, 24 h, 48 h and 72 h after introduction.

The results are expressed as previously, in FIGS. 1 and 5 to 8, respectively.

In FIGS. 5 to 8, curve C₁ represents the Gaussian distribution of the fluorescence of the sample of control cells (without dextran sulfate) (sample 1).

In FIG. 5, curve C4₁ represents the Gaussian distribution of the fluorescence of the cell sample for which the incubation was carried in the presence of 0.02 mg/ml of dextran sulfate (sample 4) for 6 h.

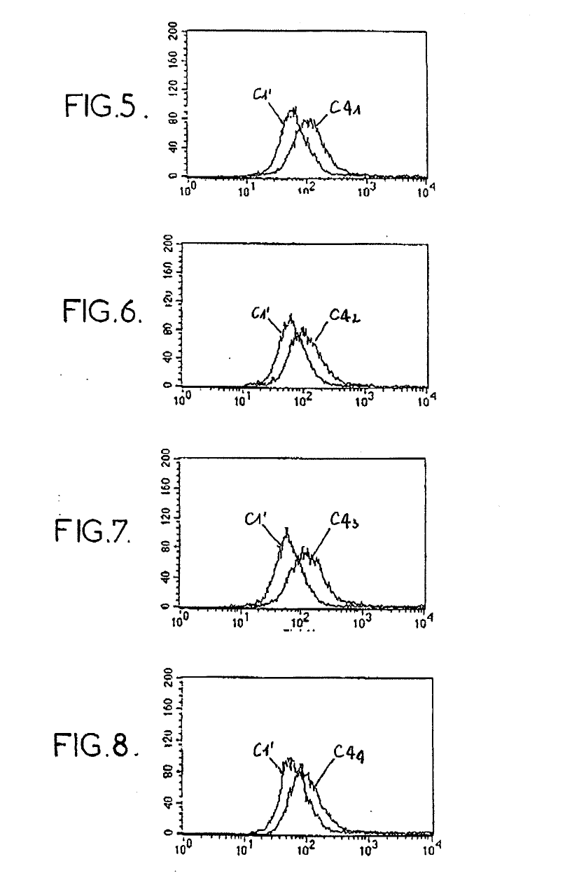

In FIG. 6, curve C4₂ represents the Gaussian distribution of the fluorescence of the cell sample for which the incubation was carried out in the presence of 0.02 mg/ml of dextran sulfate (sample 4) for 24 h.

In FIG. 7, curve C4₃ represents the Gaussian distribution of the fluorescence of the cell sample for which the incubation was carried out in the presence of 0.02 mg/ml of dextran sulfate (sample 4) for 48 h.

In FIG. 8, curve C4₄ represents the Gaussian distribution of the fluorescence of the cell sample for which the incubation was carried out in the presence of 0.02 mg/ml of dextran sulfate (sample 4) for 72 h.

The maximum values (values of the peak of each of the curves) and the corresponding ratio are given in table II below.

TABLE II

Fluorescence

| Duration | Value measured | Ratio |
|---|---|---|
| 0 (curve C_{1′}) | 50 | 1 |
| 6 h (curve C4₁) | 100 | 2 |
| 24 h (curve C4₂) | 120 | 2.4 |
| 48 h (curve C4₃) | 150 | 3 |
| 72 h (curve C4₄) | 80 | 1.6 |

Examination of these results shows that the maximum of the effect consisting of an increase in the number of Fas receptors at the cell surface is obtained after incubation for 48 h. Before this, the effect of the dextran sulfate is not at its maximum; and after 48 h, the receptors which are at the surface are degraded or return to the center of the cell.

### EXAMPLE 2

Use of an iota-carrageenan marketed by the company HERCULES.

In this example, the same procedure is carried out as in example 1 for the assays of duration of activity, but the dextran sulfate is replaced with iota-carrageenan (Ic) marketed by the company HERCULES and corresponding to formula (III) mentioned above. 0.2 mg/ml of Ic, the only concentration tested, is therefore introduced into the culture medium. A control is also prepared as in example 1, as is the corresponding ratio.

The number of Fas receptors at the surface is measured using the FACS Scan Flow Cytometer device, first 6 h, and then 24 h, 48 h and 72 h, after introduction of Ic.

Measurements were carried out at the same time on the control.

The results obtained are given in table III below; they correspond to the value of the peak of the curve of Gaussian distribution of the fluorescence, as in example 1, and also the corresponding ratio.

TABLE III

| | 6 h | | 24 h | | 48 h | | 72 h | |
|---|---|---|---|---|---|---|---|---|
| | Value | | Value | | Value | | Value | |
| | measured | Ratio | measured | Ratio | measured | Ratio | measured | Ratio |
| Control | 60 | 1 | 60 | 1 | 60 | 1 | 60 | 1 |
| Ic | 80 | 1.3 | 180 | 2.7 | 125 | 2.1 | 100 | 1.7 |
| (0.2 mg/ml) | | | | | | | | |

On examining the results given in table II, it appears that the maximum of the effect consisting of an increase in the number of Fas receptors at the cell surface is obtained after incubation for 24 h. This represents a memory effect since, if there are more receptors at the surface, the apoptosis induced by an anti-Fas receptor antibody will be more intense.

### EXAMPLE 3

The substance studied is dextran sulfate.

Work was carried out on a culture of liver cells named Hep-G2 (marketed by ECACC (European collection of human genetic cell lines)).

The same procedure is carried out as in example 1, replacing the T lymphocytes of the Jurkat type with Hep-G2 liver cells.

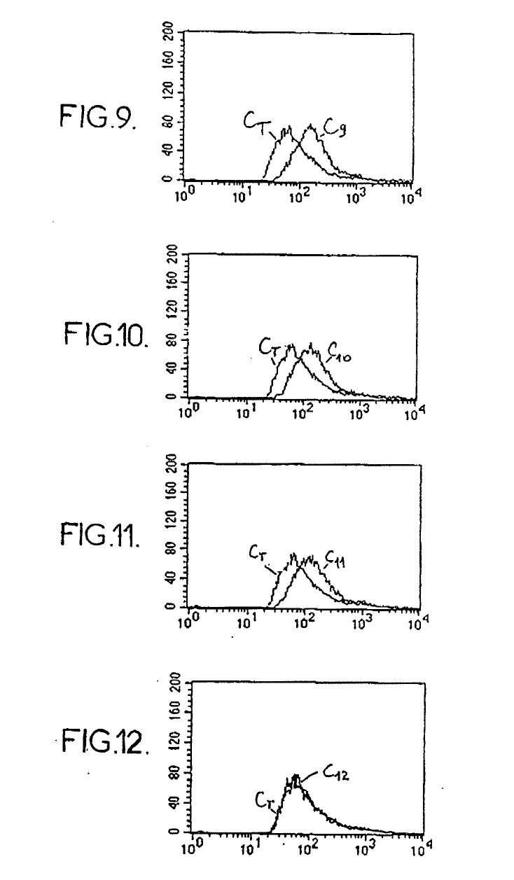

The presence of Fas receptors at the surface of the cells was measured by fluorescence, as in example 1, and the results obtained are expressed as in example 1, in the form of FIGS. 9 to 12.

In FIGS. 9 to 12, curve CT represents the Gaussian distribution of the fluorescence of the sample of control cells (without dextran sulfate) (sample 1).

In FIG. 9, curve C9 represents the Gaussian distribution of the fluorescence of the cell sample for which the incubation was carried out in the presence of 0.2 mg/ml of dextran sulfate (sample 9).

In FIG. 10, curve C10 represents the Gaussian distribution of the fluorescence of the cell sample for which the incubation was carried out in the presence of 0.1 mg/ml of dextran sulfate (sample 10).

In FIG. 11, curve C11 represents the Gaussian distribution of the fluorescence of the cell sample for which the incubation was carried out in the presence of 0.02 mg/ml of dextran sulfate (sample 11).

In FIG. 12, curve C12 represents the Gaussian distribution of the fluorescence of the cell sample for which the incubation was carried out in the presence of 0.002 mg/ml of dextran sulfate (sample 12).

The maximum values (value of the peak of each of the curves C_{T}, and C9 to C12) and the corresponding ratio are given in table IV below:

TABLE IV

| | |
|---|---|
| Concentration of | Fluorescence |

| dextran sulfate | Value measured | Ratio |
|---|---|---|
| 0 (curve C_{T}) | 60 | 1 |
| 0.2 mg/ml (curve C9) | 160 | 2.7 |
| 0.1 mg/ml (curve C10) | 140 | 2.3 |
| 0.02 mg/ml (curve C11) | 120 | 2 |
| 0.002 mg/ml (curve C12) | 60 | 1 |

On examining the results given in table IV, it appears that the minimum concentration of dextran sulfate is 0.02 mg/ml, that at a greater dilution the effect is too diminished, and that the optimum concentration is 0.1 mg/ml.

The effectiveness of the dextran sulfate on the Hep-G2 liver cells is similar to that encountered on the T lymphocytes of the Jurkat type.

A further series of assays was then carried out in order to measure the duration of the effect consisting of an increase in the number of Fas receptors at the cell surface caused by 0.2 mg/ml of dextran sulfate.

For this, the same procedure as in example 1 was carried out.

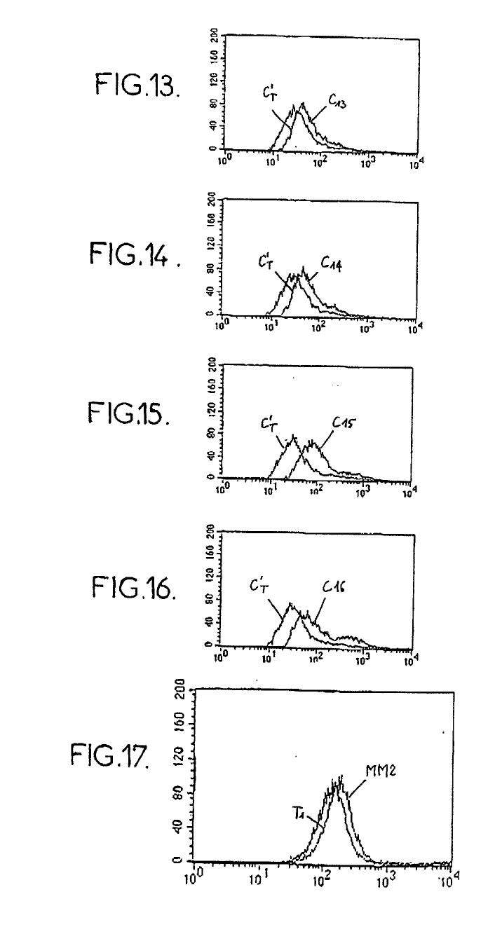

The results are expressed in FIGS. 13 to 16, respectively.

In FIGS. 13 to 16, curve C′_{T} represents the Gaussian distribution of the fluorescence of the sample of control cells (without dextran sulfate).

In FIG. 13, curve C13 represents the Gaussian distribution of the fluorescence of the cell sample for which the incubation was carried out in the presence of 0.02 mg/ml of dextran sulfate (sample 13) for 6 h.

In FIG. 14, curve C14 represents the Gaussian distribution of the fluorescence of the cell sample for which the incubation was carried out in the presence of 0.02 mg/ml of dextran sulfate (sample 14) for 24 h.

In FIG. 15, curve C15 represents the Gaussian distribution of the fluorescence of the cell sample for which the incubation was carried out in the presence of 0.02 mg/ml of dextran sulfate (sample 15) for 48 h.

In FIG. 16, curve C16 represents the Gaussian distribution of the fluorescence of the cell sample for which the incubation was carried out in the presence of 0.02 mg/ml of dextran sulfate (sample 16) for 72 h.

The maximum values (values of the peak of each of the curves) and also the corresponding ratio are given in table V below.

TABLE V

Fluorescence

| Duration | Value measured | Ratio |
|---|---|---|
| 0 (curve C′_{T}) | 25 | 1 |
| 6 h (curve C13) | 40 | 1.6 |
| 24 h (curve C14) | 50 | 2 |
| 48 h (curve C15) | 70 | 2.8 |
| 72 h (curve C16) | 50 | 2 |

On examining the results given in table V, it appears that the maximum of the effect consisting of an increase in the number of Fas receptors at the cell surface is obtained after incubation for 48 h. This represents the memory effect since, if there are more receptors at the surface, the apoptosis induced by an Fas agonist antibody will be more intense.

### EXAMPLE 4

In this example, the effect of dextran sulfate at a concentration of 0.1 mg/ml on the increase in the number of TNF alpha receptors on Jurkat-type cells was measured.

The medium used for culturing the Jurkat-type cells is that marketed by the company Life Technologies under the name “RPMI 1640 medium”; this medium is described by Moore et al. in the publication “A.M.A.” 199, 519 (1967).

10⁶ T lymphocytes are suspended in 5 ml of this medium. The culturing was carried out in an incubator at a temperature of 37° C. and in an atmosphere containing 5% CO₂. After incubation for 24 hours, the cells multiplied to reach 2×10⁶ cells.

These cells are recovered by centrifugation, and are washed in an isotonic PBS buffer. A further incubation is then carried out in 1 ml of the abovementioned medium at 4° C. and in an atmosphere containing 5% CO₂, for 45 minutes in the presence of an anti-human p55 TNF receptor, clone MR1-2, ref. MON9062 produced in mice by Genzyme SA, diluted to 1/50 in PBS containing 1% of bovine serum albumin (BSA).

The cells are recovered and are washed with the isotonic PBS buffer. A further incubation is carried out with an antibody against mouse immunoglobulins (IgGs) (produced in the goat) coupled to fluorescein isothiocyanate, diluted to 1/50 in PBS containing 1% of BSA.

Controls were also prepared by incubating the cells only with the second antibody coupled to fluorescein isothiocyanate.

The influence of the dextran sulfate is determined by introducing 0.1 mg of dextran sulfate per ml into the culture medium.

The presence of TNF alpha receptors at the surface of the cells was measured by fluorescence as in the previous examples.

In FIG. 17, in which the Y-axis represents the number (to within one multiplication factor) of TNF alpha receptors at the surface of the cells, and in which the X-axis represents the intensity of fluorescence (arbitrary units), the Gaussian distribution of the fluorescence for the control sample (curve T1) and for the sample which was incubated in the presence of 0.1 mg/ml of dextran sulfate (curve C17) is represented in the form of a curve.

The maximum values (values of the peak of each of the curves) and also the corresponding ratio are given in table VI below:

TABLE VI

| | |
|---|---|
| Concentration of | Fluorescence |

| dextran sulfate | Value measured | Ratio |
|---|---|---|
| 0 | 175 | 1 |
| 0.1 mg/ml | 200 | 1.14 |

The effect consisting of an increase in the number of TNF alpha receptors at the surface is weak (10% increase).

On the basis of the teachings provided by examples 1 to 4, it is possible to develop medicines capable of activating apoptosis by increasing the number of apoptosis receptors at the cell surface, by making them comprise, as active principle, a polysaccharide substance as defined above, preferably a dextran sulfate, optionally in combination with an anti-apoptosis receptor antibody.

The amount of active principle administered per day is approximately 0.1 to 0.01 mg/kg of body weight.

The medicines in question are advantageously provided in the form of an aerosol, an injectable solution, a presentation for topical application such as ointments, gels, etc.

They may be administered by inhalation, orally, or by application to the skin.

These medicines make it possible to obtain activation or stimulation of apoptosis in the case of disorders of the group comprising autoimmune diseases, cancers and asthma, and also when, subsequent to damage to or modification of the DNA of skin cells subjected to UV radiation, thymidine dimers or 8-oxo-guanosine dimers in particular form, due to mutations in the DNA, these being mutations which may trigger the appearance of tumors.

By way of examples, given below are possible compositions of an ointment, injectable solution, aerosol and aftersun milk illustrating uses in accordance with the invention.

In the following examples, the polysaccharide substance used is iota-carrageenan, Ic.

### EXAMPLE 5

### Ointment

| | |
|---|---|
| Lanolin | 36 g |
| Petroleum jelly | 36 g |
| Sweet almond oil | 22 g |
| Zinc oxide | 5 g |
| Ic | 1 g |

### EXAMPLE 6

### Injectable Solution

| | |
|---|---|
| Ic | 0.006 g |
| Water for injectable preparation | qs 10 ml |

### EXAMPLE 7

### Solution for Aerosol

| | |
|---|---|
| Ic | 0.25 mg |
| Sodium edetate | 2.5 mg |
| Sodium hydroxide | qs pH = 7 |
| Water for injectable preparation | qs 5 ml |

### EXAMPLE 8

### Aftersun Milk

| | (%) |
|---|---|
| PEG-30 dipolyhydroxystearate (Arlacel P135) | 2.0 |
| Cyclomethicone and PPG-15 stearyl ether | |
| (Arlamol S7) | 6.0 |
| Isohexadecane (Arlamol HD) | 12.0 |
| Sorbeth-30 (Atlas G-2330) | 4.0 |
| Magnesium sulfate heptahydrate | 0.7 |
| Ic | 0.2 |
| Bisabolol | 0.2 |
| Demineralized water | 73.8 |
| Mixture of propylene glycol, diazolidinylurea, | 1.0 |
| methylparaben and propylparaben (Germaben II) | |
| Fragrance | 0.1 |

1. The use of at least one polysaccharide substance having at least five saccharide units and comprising, on at least some of its individual units, at least one substituent carrying at least one negative charge, chosen from the group comprising in particular sulfate, acetate, phosphate and phosphonate groups, for preparing a medicine for activating apoptosis by increasing the number of apoptosis receptors at the cell surface, said receptors being those of the group comprising Fas, TNF, TRAIL and CD40 receptors, preferably Fas receptors.

2. The use as claimed in claim 1, of a polysaccharide substance of the group comprising:
the oligosaccharides derived, by enzymatic or chemical process, from the polymers of the group comprising β-1,3 glucans optionally comprising β-1,6 branchings, and comprising, on at least some of their individual units, at least one substituent carrying at least one negative charge, chosen from the group comprising in particular sulfate, acetate, phosphate and phosphonate groups,
the oligosaccharides derived, by enzymatic or chemical process, from sulfated galactans, in particular carrageenans, agars and porphyrans.

3. The use as claimed in claim 1, of a polysaccharide substance corresponding to the formula 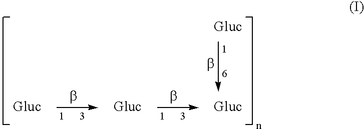
in which Gluc represents glucose and n represents an integer from 1 to 50, preferably from 5 to 10, and in which the number of branches varies from 0 to 3 per repeat unit, and comprises, on at least some of its individual units, at least one substituent carrying at least one negative charge, chosen from the group comprising in particular sulfate, acetate and phosphate groups.

4. The use as claimed in claim 1, of a polysaccharide substance which is a repeat disaccharide corresponding to the formula 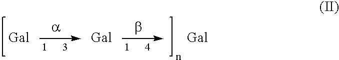
in which Gal represents galactose and n represents an integer from 1 to 50, preferably from 1 to 20, at least some of the repeat disaccharides of formula (II) possibly comprising one or more sulfate groups.

5. The use as claimed in claim 1, of dextran comprising, on at least some of its individual units, at least one substituent carrying at least one negative charge, chosen from the group comprising in particular sulfate, acetate, phosphate and phosphonate groups, preferably of sulfated dextran, and even more preferentially of the sulfated dextran marketed by the company SIGMA.

6. The use as claimed in claim 1, of a natural polysaccharide extracted from red alga, of the iota-carrageenan type, marketed by the company HERCULES and having the formula 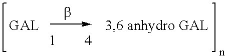
in which:
GAL represents galactose,
3,6 anhydro GAL represents 3,6 anhydrogalactose linked via a β1-4 linkage, and
n represents an integer from 2 to 300, preferably from 2 to 200,
the repeat disaccharides possibly carrying 2 sulfate esters.

7. The use as claimed in one of claims 1 to 6, of a polysaccharide substance in combination with an anti-apoptosis receptor antibody, for preparing a medicine for activating apoptosis by increasing the number of apoptosis receptors at the cell surface.

8. The use as claimed in one of claims 1 to 7, for preparing a medicine for treating diseases belonging to the group of autoimmune diseases, cancers and asthma.

9. The use as claimed in one of claims 1 to 8, of a polysaccharide substance, for preparing a medicine provided in the form of an aerosol.

10. The use as claimed in one of claims 1 to 9, of 0.1 to 0.01 mg/kg of body weight of the polysaccharide substance.

11. A medicine characterized in that it comprises an effective amount of at least one polysaccharide substance as used in any one of claims 1 to 6, and in that it can be administered by inhalation.

12. An aerosol characterized in that it comprises an effective amount of at least one polysaccharide substance as used in any one of claims 1 to 6.

13. A medicine characterized in that it comprises an effective amount of at least one polysaccharide substance as used in any one of claims 1 to 6, and in that it can be administered topically, especially on the skin, and in particular in the form of an ointment or of a gel.
